(19) United States
(12) Patent Application Publication
(10) Pub. No.: US 2009/0304747 A1
(43) Pub. Date: Dec. 10, 2009

(54) USE OF DMSO AND BOTULINUM TOXIN THERAPY FOR URINARY INCONTINENCE AND RELATED DISORDERS

(75) Inventor: Steven P. Petrou, Ponte Vedra Beach, FL (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/300,698

(22) PCT Filed: May 9, 2007
(86) PCT No.: PCT/US2007/068555

§ 371 Date: Jun. 25, 2009

Related U.S. Application Data
(60) Provisional application No. 60/800,645, filed on May 16, 2006.

(51) Int. Cl. A61K 39/08

(52) U.S. Cl. 424/239.1

(57) ABSTRACT

This disclosure provides a method of treating urologic and related disorders, for example, urinary incontinence, in women through the administration of a composition comprising a neurotoxin and DMSO. Methods for making and using the described compositions are also provided.

### TECHNICAL FIELD

This invention relates to a method for the treatment of urinary voiding dysfunction, for example, urinary incontinence, and more particularly to the treatment of overactive bladder and detrusor hyperreflexia in women.

### BACKGROUND

Upwards of 17% of the population of the United States suffers from overactive bladder (OAB). In 2000, OAB had a total economic cost of over 12 billion dollars, of which over 7 billion dollars was attributed to females. Current treatment for women with detrusor hyperreflexia (DH), detrusor overactivity (DO), or OAB consists of anticholinergic medications that partially block parasympathetic innervation of the detrusor muscle. These medications may have troublesome side effects and variable efficacy.

Women with signs and symptoms of OAB pose a difficult therapeutic challenge if anticholinergic medication fails. They must either live with their malady or undergo open surgery such as sacral root rhizotomy, augmentation, enterocystoplasty, percutaneous neuromodulation, stimulator implantation, or urinary diversion.

In response to this clinical dilemma, the use of botulinum toxin injections have been implemented in the treatment of lower urinary tract dysfunction. Currently, botulinum-A toxin has been investigated as a safe and reversible alternative to open surgery in patients failing anticholinergic therapy. Generally, administration requires the use of a cystoscope, injection needle, and aesthesia. The toxin is delivered with a needle through the scope puncturing the bladder lining into the bladder wall 20-30 times. Preliminary research suggests that botulinum-A toxin destrusor injections can suppress bladder overactivity and increase bladder filling. See, e.g., U.S. Pat. Nos. 6,667,041 and 7,001,602. A treatment method that is capable of delivering the botulinum toxin without the potential complications associated with surgical procedures or the need for local, regional, or general anesthesia is desirable.

### SUMMARY

Accordingly, the disclosure provided herein relates to a method of treating the symptoms of urinary incontinence through the administration of a composition comprising dimethyl sulfoxide (DMSO) and a neurotoxin such as botulinum toxin. The composition can be in solution (e.g., an aqueous solution). The botulinum toxin can be botulinum-A toxin and can be present at a concentration of from about 100 to about 400 units. The DMSO can be present from about 20 to about 90% by weight (e.g., about 50% by weight).

In one aspect of the disclosure, a method of treating, preventing, or ameliorating one or more symptoms associated with urinary incontinence (e.g., overactive bladder, detrusor hyperreflexia, and detrusor overactivity) in a mammal is provided. A mammal may be, for example, a human, monkey, dog, cat, horse, sheep, cow, or pig. In certain embodiments, the mammal is a human female. The method includes the administration of a therapeutically effective amount of a composition comprising DMSO and a neurotoxin (e.g., botulinum toxin) thereby treating, preventing, or ameliorating one or more of the symptoms of urinary incontinence in said mammal. The symptoms and signs of urinary incontinence may be those commonly associated with OAB, DH, DO, urinary urgency, or urinary urge incontinence.

The composition administered to the mammal may include DMSO present in an aqueous solution from about 20% to about 90% by weight (e.g., about 25% to about 90%, or about 50% by weight), and botulinum toxin present from about 100 to about 400 units (e.g., about 300 units). In some embodiments, the botulinum toxin may be botulinum-A toxin (Botox). In certain embodiments, the composition can be administered to the bladder of the mammal. The composition can be administered directly into the bladder of a mammal through the use of a non-needle delivery system, e.g., direct instillation into the bladder. Administration of this sort may be accomplished through the use of a catheter.

The invention also features a method of treating, preventing, or ameliorating one or more symptoms associated with urinary incontinence in a mammal (e.g., a human such as a human female). The method includes administrating to the mammal a therapeutically effective amount of a composition of matter that includes a 50% by weight solution of DMSO containing 300 units of botulinum toxin (e.g., botulinum A toxin), thereby treating, preventing, or ameliorating one or more of the symptoms of urinary incontinence in the mammal. Administration of the composition can result in increased bladder capacity in said mammal. The composition can be administered through the use of a catheter (e.g., direct instillation into the bladder).

In another aspect of the present disclosure, a therapeutically effective amount of the DMSO/neurotoxin composition will be determined by the reduction in urinary incontinence symptoms. This reduction may be indicated by increased bladder capacity and reduction in urinary incontinence episodes.

Unless otherwise defined, all technical and scientific terms used herein have the same meaning as commonly understood by one of ordinary skill in the art to which this invention belongs. Although methods and materials similar or equivalent to those described herein can be used in practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DETAILED DESCRIPTION

Urinary control relies on the coordinated activities of the smooth muscle tissue of the urethra and bladder, skeletal muscle, voluntary inhibition, and the somatic and autonomic nervous system. Urinary incontinence is the inability to control the release of urine from your bladder. It can result from anatomic, physiologic, or pathologic factors. More than 13 million people in the United States experience incontinence. While older women constitute the majority of people who suffer from urinary incontinence, both women and men, young and old, are affected by the disorder.

There are five major types of urinary incontinence: (1) stress incontinence, in which loss of urine is triggered by a stress event (e.g., laughing, coughing, sneezing, lifting heavy objects, or exercising); (2) urge incontinence, which is characterized by a sudden, intense urge to urinate, followed by an involuntary loss of urine; (3) overflow incontinence, wherein the bladder is unable to fully expel its contents, which leaves the bladder continually full and causes urine to dribble out; (4) mixed incontinence, in which more than one of the above symptoms are observed; and (5) functional incontinence, which is characterized by a normal control over urinary functions, but wherein some other disorder, such as a physical or mental impairment, prevents arrival to the toilet in time. Many conditions may contribute to the above symptoms, including diabetes, pregnancy, aging, Alzheimer's, weight gain, urinary tract infections, side effects from medications, depression, brain and spinal cord injuries, multiple sclerosis, or stroke. Urinary incontinence is often referred to as overactive bladder (OAB), detrusor overactivity (DO), and detrusor hyperreflexia (DH).

There are currently many methods of treating urinary incontinence, some of which are met with greater success than others. One such treatment method includes behavioral techniques, including, for example, bladder training, pelvic muscle exercises, biofeedback, electrical stimulation, diet management, or timed voiding. In some cases, medications have been found to control urinary incontinence. Such medications can include anticholinergic, or antispasmodic medications, antidepressants (e.g., Imipramine), antibiotics, or hormone replacement. Often, these medications have side effects or are found to be ineffective in some patients. When this occurs, use of a medical device (e.g., urethral insert or pessary nerve stimulator) may be prescribed, or surgery (e.g., sachral root rhizotomy, augmentation, enterocystoplasty, permanent implant nerve stimulator, or urinary diversion) may be required.

### Compositions of Matter

Provided herein are methods and compositions for treating, preventing, or ameliorating one or more symptoms associated with urinary voiding dysfunction, e.g., urinary incontinence, in a mammal. The methods can include administrating a therapeutically effective amount of a composition comprising DMSO and a neurotoxin to a mammal.

Preferably, the neurotoxin in the composition is highly selective, easy to deliver, and safe. Non-limiting examples of neurotoxin agents include botulinum toxin, saxitoxin, tetanus toxin, capsaicin, resiniferatoxin, α-bungarotoxin, and tetrodotoxin. Suitable botulinum toxins include, for example, botulinum toxins A, B, C (C1 and C2), D, E, F, or G. Botulinum toxins A, B, and F are particularly useful.

Botulinum-A toxin is the most potent biological toxin known; it produces temporary muscular paralysis when injected locally. The toxin acts as a selective inhibitor of acetylcholine release from pre-synaptic nerve endings. Botulinum-A toxin has been FDA approved since the 1980s for the treatment of focal dystonias such as blepharospasm, nondystonic disorders such as hemifacial spasms, disorders of conjugate eye movement such as strabismus and nystagmus, spasticity disorders such as multiple sclerosis and cerebral palsy, and for disorders of localized muscle spasm. In addition, botulinum toxin A has been used to treat age related rhytids of the upper face. Botulinum toxin A is safe and effective to use, and is relatively painless with rare side effects characterized as mild and transient. Onset of action takes place within 24 to 72 hours after injection and lasts 2 to 6 months. Botulinum toxin A is available commercially, e.g., from Allergan, Inc. (Irvine, Calif., Botox®) and Speywood Pharmaceuticals (England, Dysport®). See, e.g., National Institutes of Health Consensus Development Conference Statement, Nov. 12-14, 1990. Arch. Neurol., 1991, 48:1294; Jankovic, J. and Schwartx, K., Neurology, 1993, 43: 834; and Pierson, S., Katz, D., Torsy, D., Arch. Phys. Med. Rehabil., 1996, 77:717.

Dosages of botulinum toxin A required for local immobilization typically do not exceed 1 unit toxin per kg body weight and are safe. Primate studies have indicated that no systemic effects are observed at dosages below 33 units/kg body weight. See, for example, Scott and Suzuki, Mov. Disord., 1988, 3:333-335.

Botulinum toxins also can be obtained by purifying the toxins from strains of Clostridium botulinum, using standard techniques. For example, botulinum toxin A can be produced in a Hall strain using a nutritive medium containing casein digest, yeast extract, and dextrose. After lysis of the culture, the toxin is released into the medium and activated by proteases, and then is acid precipitated. Further purification can include extraction with a sodium phosphate buffer, ethanol precipitation, and crystallization in ammonium sulfate. See, for example, Schantz, E. J. and Johnson, E. A., Microbiol. Rev., 1992, 56(1):80-99.

DMSO is a widely used solvent with pharmacological actions including bacteriostatic activity, analgesia, vasodilation, diuresis, and muscle relaxation. It is well known in the urologic literature as a non-specific anti-inflammatory agent that can penetrate/permeate the glycosaminoglycan layer of the bladder with direct effect upon the detrusor muscle. Following topical application, DMSO is absorbed and generally distributed in the tissues and body fluids. See, e.g., Chen, D., Song, D., Wientjes, G., and Au, J. L-S., Clin. Cancer Res., 2003, 9:363-369. Because of its mechanism of action, its current use in Urology is FDA approved for bladder inflammatory disorders such as interstitial cystitis.

In one embodiment, the composition includes botulinum-A toxin and DMSO in solution, e.g., aqueous solution. This solution may include from about 100 to about 600 units of botulinum-A toxin (e.g., 100, 125, 150, 175, 200, 225, 250, 275, 300, 325, 350, 375, 400, 425, 450, 475, 500, 525, 550, or 575 units) and from about 20 to about 90% w/w solution of DMSO (e.g., 20%, 25%, 30%, 35%, 40%, 45%, 50%, 55%, 60%, 65%, 70%, 75%, 80%, 85%, or 90%). In some embodiments, the solution includes 50% w/w solution of DMSO and 300 units of botulinum-A toxin. The two components can be combined and stored, e.g., at 4° C., until used, or mixed prior to administration. For example, prior to administration, an appropriate amount of lyophilized botulinum-A toxin can be reconstituted with an appropriate solution of DMSO to result in a solution containing the desired final concentration of botulinum-A and DMSO.

In certain embodiments, the portion of the composition comprising DMSO may include, for example, one or more of water, saline, aqueous dextrose, glycerol, glycols, ethanol, and the like, to thereby form a solution or suspension. If desired, the composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

The compositions described above can be formulated as articles of manufacture, e.g., kits, containing the described compositions or components for preparing the described compositions. For example, in one embodiment, a kit may include separate containers, e.g., vials, of DMSO and botulinum-A, for mixing prior to administration. In other embodiments, previously prepared solutions of DMSO and botulinum-A toxin may be included in a kit. Additional items such as packaging, labels including instructions, syringes, catheters, and other equipment necessary or useful for administration can be included.

The compositions described above are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active composition sufficient to produce the desired therapeutic effect, in association with any required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, capsules, or bottles of pints or gallons. Hence, multiple dose form is a multiple of unit-doses which are not segregated in packaging.

Actual methods of preparing such dosage forms are known, or will be apparent, to those skilled in this art; for example, see Remington's Pharmaceutical Sciences, Mack Publishing Company, Easton, Pa., 15th Edition, 1975.

### Methods of Use

The previously described compositions of neurotoxin and DMSO can be used to treat, prevent, or ameliorate one or more symptoms associated with urinary incontinence in a mammal (e.g., a human female). In certain aspects of this application, administration of the Botox/DMSO composition may include administering an amount of about 10 to about 100 mL (e.g., 10, 20, 30, 40, 50, 60, 70, 80, 90, or 100 mL) of a solution including from about 100 to about 400 units of botulinum-A toxin and from about 20 to about 90% w/w solution of DMSO.

Current administration of botulinum-A toxin utilizes cystoscopic guided injections into the bladder lining and/or muscle. While no permanent systemic side effects have been noted, there is transient injection site discomfort associated with the process and it requires the use of anesthesia. In certain embodiments, the composition comprising botulinum-A toxin and DMSO may be delivered into the bladder through guided injections into the bladder. In other embodiments of the present method, the composition of botulinum-A toxin and DMSO is delivered through a catheter based delivery system (e.g., direct instillation into the bladder). This latter delivery method may limit the potential complications associated with surgical procedures or anesthesia.

A catheter delivery system is an effective means to deliver medications directly into the bladder. The use of oral medications are limited by the circulating concentration of the drug in the blood stream, but treatment delivery through the use of urinary bladder instillation allows for direct pumping of a therapeutic composition into the bladder through a urethral catheter. The composition, e.g., DMSO and botulinum-A toxin, is held in the bladder for a “dwell time” before the bladder is voided. This procedure allows the treatment of the urinary bladder wall directly with high concentrations of medicine.

As used herein, treatment means any manner in which one or more of the symptoms of a disease or disorder are ameliorated or otherwise beneficially altered. Treatment also encompasses any pharmaceutical use of the compositions herein, such as use for treating diseases or disorders in which the botulinum-A toxin/DMSO formulation is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

The active ingredient may be administered at once, or may be metachronously dosed, e.g., to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

To evaluate the effectiveness of a urinary incontinence treatment, improvements to the patient's symptoms can be measured by the use of accepted measurement protocols, such as: (1) the Blaivas-Groutz anti-incontinence score or (2) the Indevus Urgency Severity Scale (IUSS). Briefly, the Blaivas-Goutz score combines information regarding the number of incontinence episodes in a 24-hour period, measurements of 24-hour pad weights, and a qualitative rating by the patient into a score ranging from 0 to 6. Similarly, the IUSS scoring system represents a qualitative assessment by the patient regarding the severity of the incontinence that is then converted to a score from 0 to 3. Each scoring system has been validated as an accurate and reliable means of determining the severity of urinary incontinence.

The invention is further described in the following examples, which do not limit the scope of the invention described in the claims.

### EXAMPLES

### Example 1

A composition consisting of botulinum-A toxin and DMSO is prepared by combining 200 units of botulinum toxin Type A and 50 mL of dimethyl sulfoxide in a 50% w/w aqueous solution.

### Example 2

A composition consisting of botulinum-A toxin and DMSO is prepared by combining 300 units of botulinum toxin Type A and 50 mL of dimethyl sulfoxide in a 50% w/w aqueous solution.

### Example 3

A group of three female patients suffering from symptoms of urinary incontinence will undergo bladder instillation of 50 mL of the composition as described in Example 1. Patients will be chosen based on the results of baseline urodynamic studies and cystoscopy which will be used to establish detrusor hyperreflexia or detrusor overactivity, while ruling out stress urinary incontinence. The patients will undergo an initial examination consisting of a pelvic exam, a urine analysis and culture determination, and a post void urinary residual.

The composition will be administered using sterile techniques and 1% lidocaine gel. A 16F catheter will be introduced per urethra and the bladder drained of any urine. The composition will be instilled with gravity pressure, and the catheter removed. The patient will retain the composition as long as possible and then spontaneously void the composition in a convenient commode. Patients will be monitored 24 hours and one week after the initial instillation and then at post-procedure intervals of 1 and 3 months.

Effectiveness of the composition will be evaluated using administration of Blaivas-Goutz and IUSS scoring systems as well as a urine culture, a post void residual test, and urine analysis.

### Example 4

A similar study, as detailed in Example 3, will be conducted. A group of 25female patients with symptoms of urinary incontinence will undergo bladder instillation of the composition as described in Example 2. The initial examination, administration, and evaluation of this study will be conducted as described above.

A number of embodiments of the invention have been described. Nevertheless, it will be understood that various modifications may be made without departing from the spirit and scope of the invention. Accordingly, other embodiments are within the scope of the following claims.

What is claimed is:

1. A composition of matter comprising botulinum toxin and DMSO.

2. The composition of claim 1, wherein said composition is a solution.

3. The composition of claim 1, wherein said composition is an aqueous solution.

4. The composition of claim 1, wherein said botulinum toxin is botulinum-A toxin.

5. The composition of claim 4, wherein said botulinum-A toxin is present at a concentration of from about 100 to about 400 units.

6. The composition in claim 1, wherein said DMSO is present from about 20 to about 90% by weight.

7. The composition of claim 6, wherein said DMSO is present at about 50% by weight.

8. A method of treating, preventing, or ameliorating one or more symptoms associated with urinary incontinence in a mammal comprising administrating to said mammal a therapeutically effective amount of a composition of matter comprising DMSO and botulinum toxin, thereby treating, preventing, or ameliorating one or more of the symptoms of urinary incontinence in said mammal.

9. The method of claim 8, wherein said mammal is a human.

10. The method of claim 8, wherein said mammal is a human female.

11. The method of claim 8, wherein said botulinum toxin is botulinum-A toxin.

12. The method of claim 8, wherein said DMSO is present at about 50% by weight.

13. The method of claim 8, wherein said urinary incontinence is selected from overactive bladder, detrusor hyperreflexia, and detrusor overactivity.

14. The method of claim 8, wherein said composition of matter comprising DMSO and botulinum toxin is administered to the bladder of said mammal.

15. The method of claim 14, wherein said composition of matter comprising DMSO and botulinum toxin is administered through the use of a catheter.

16. The method of claim 15, wherein said composition of matter comprising DMSO and botulinum toxin is directly instilled into the bladder.

17. A method of treating, preventing, or ameliorating one or more symptoms associated with urinary incontinence in a mammal comprising administrating to said mammal a therapeutically effective amount of a composition of matter comprising a 50% by weight solution of DMSO containing 300 units of botulinum toxin, thereby treating, preventing, or ameliorating one or more of the symptoms of urinary incontinence in said mammal.

18. The method of claim 17, wherein said mammal is a human.

19. The method of claim 17, wherein said mammal is a human female.

20. The method of claim 17, wherein said botulinum toxin is botulinum-A toxin.

21. The method of claim 17, wherein administration of said composition of matter comprising DMSO and botulinum toxin results in increased bladder capacity in said mammal.

22. The method of claim 17, wherein said composition of matter comprising DMSO and botulinum toxin is administered through the use of a catheter.

23. The method of claim 22, wherein said composition of matter comprising DMSO and botulinum toxin is directly instilled into the bladder.
